(19) United States
(12) Patent Application Publication
(10) Pub. No.: US 2026/0222679 A1
(43) Pub. Date: Jul. 30, 2026

(54) IMAGING APPARATUS HAVING A SPLIT IMAGE COMPONENT

(71) Applicant: Identifeye Health Inc., Guilford, CT (US)

(72) Inventors: Paul E. Glenn, Wellesley, MA (US); Christopher Thomas McNulty, Guilford, CT (US); Georg Schuele, Portola Valley, CA (US); Mark M. Meyers, Madison, CT (US); Luis Gerardo Benavides Leal, New Haven, CT (US)

(21) Appl. No.: 19/036,954

(22) Filed: Jan. 24, 2025

Related U.S. Application Data
(60) Provisional application No. 63/625,075, filed on Jan. 25, 2024.

(51) Int. Cl. H04N 23/60; G06T 7/70; H04N 23/56

(52) U.S. Cl. CPC H04N 23/64; G06T 7/70; H04N 23/56; G06T 2207/30041

(57) ABSTRACT

Some aspects relate to techniques for localizing a subject's pupil in a field of view of a first imaging device in response to receiving light transmitted through a split image component of the first imaging device. Some aspects relate to an imaging apparatus comprising a split image component, wherein a first imaging device of the imaging apparatus is configured to localize a subject's pupil in a field of view of the imaging device in response to transmission of light through the split image component.

### CROSS-REFERENCE TO RELATED APPLICATIONS

This application claims the benefit of priority under 35 U.S.C. § 119 (e) of U.S. Provisional Patent Application No. 63/625,075, titled “IMAGING APPARATUS HAVING A SPLIT IMAGE COMPONENT,” filed Jan. 25, 2024, which is incorporated by reference herein in its entirety.

### FIELD OF THE DISCLOSURE

The present disclosure relates to techniques for imaging and/or measuring a subject's eye, including the subject's retina fundus.

### BACKGROUND

Techniques for imaging and/or measuring a subject's eye would benefit from improvement.

### SUMMARY

Some aspects of the present disclosure relate to techniques comprising localizing a pupil of a subject's eye in a field of view of a pupil imaging device using an image of at least a portion of the pupil, wherein the image was generated using light that was transmitted from the pupil through a split image component of the pupil imaging device.

Some aspects of the present disclosure relate to an imaging apparatus comprising a pupil imaging device comprising a split image component, wherein the pupil imaging device is configured to localize a pupil of a subject's eye in a field of view of the pupil imaging device using an image of at least a portion of the pupil, the image having been generated using light that was transmitted from the pupil through the split image component.

The foregoing summary is not intended to be limiting. Moreover, various aspects of the present disclosure may be implemented alone or in combination with other aspects.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a schematic view of exemplary components of an imaging system having a prism configuration, according to some embodiments.

FIG. 1B is a diagram of an example prism configuration, according to some embodiments.

FIG. 2 is a block diagram of an example system 200 for localizing an imaging target, according to some embodiments.

FIG. 3A is a flowchart of an illustrative process for localizing a subject's pupil, according to some embodiments.

FIG. 3B is a flowchart of an illustrative process for determining a displacement between a current position of an imaging target and a target position, according to some embodiments.

FIG. 4 shows an example of displacement between a current position and a target position of an imaging target, according to some embodiments of the technology described herein.

FIG. 5A shows example images generated using light that has been transmitted through a split image component of an imaging device, according to some embodiments.

FIG. 5B shows an example of processing an image to determine a displacement between a current position and a target position of an imaging target, according to some embodiments.

FIG. 6A is a top perspective view of an exemplary imaging apparatus having a prism configuration, according to some embodiments.

FIG. 6B is a front perspective view of the exemplary imaging apparatus of FIG. 6A, according to some embodiments.

FIG. 6C is a side cross-sectional view of the exemplary imaging apparatus of FIG. 6A, according to some embodiments.

FIG. 6D is a top cross-sectional view of the exemplary imaging apparatus of FIG. 6A, according to some embodiments.

FIG. 6E is a front perspective view of the exemplary imaging apparatus of FIG. 6A, according to some embodiments.

FIG. 6F is a bottom perspective view of the exemplary imaging apparatus of FIG. 6A, according to some embodiments.

FIG. 7 is a schematic diagram of an illustrative computing device with which aspects described herein may be implemented.

### DETAILED DESCRIPTION

Aspects of the present disclosure provide improved techniques to assist in imaging a target (e.g., an eye) that are suitable for use in an imaging apparatus operated by a user (e.g., the subject, a clinician, a technician, a doctor, etc.). In some embodiments, the imaging apparatus includes one or more imaging devices including at least a first imaging device (e.g., a pupil imaging device) and a second imaging device. In some embodiments, the imaging apparatus further includes a split image component configured to (a) receive light from the imaging target, (b) direct a first portion of the light along a first imaging path, and (c) direct a second portion of the light along a second imaging path (e.g., at an angle relative to the first imaging path). For example, the split image component may be a split prism configuration or a split mirror configuration.

In some embodiments, the techniques include localizing the imaging target in the field of view of the first imaging device in response to receiving light transmitted through the split image component. For example, the imaging target may be localized using an image generated using the received light. In some embodiments, results of localizing the imaging target may be used to adjust a position of the imaging apparatus with respect to the subject, or to adjust a position of the imaging target with respect to the imaging apparatus, to improve the quality of the imaging.

Imaging techniques and apparatuses described herein provide medical grade imaging quality and may be produced or conducted by an untrained operator (e.g., the subject being imaged) even in the absence of a clinician or technician, thereby improving access to medical grade imaging. As described herein, a medical grade image may be an image of a part of a subject's anatomy (e.g., the subject's eye) that is useful for determining and/or diagnosing a health condition and/or disease of the subject.

Devices for imaging a target (e.g., a subject's eye) require precise positioning of the subject and the device with respect to one another. For example, the target should be positioned with respect to the imaging device such that it is substantially centered and correctly spaced along the planned beam path of the imaging device. When not positioned properly, the target may be decentered or incorrectly spaced, leading to degraded image quality.

Conventional techniques for positioning a subject and device with respect to one another are inefficient and resource intensive. Such conventional techniques require a highly skilled operator (e.g., a clinician or technician) to decide how the target (e.g., the subject's eye) and/or device should be positioned with respect to one another. For example, the operator may instruct the subject where and how to position themself before, during, and after the imaging. Additionally, the operator may then adjust the imager to obtain greater precision to optimize the target location and overlap of the cameras beam with the patient's eye. When the operator is satisfied with the subject's positioning, an image may be obtained using the device. The operator may then evaluate the obtained image, determine whether to correct the positioning of the subject and/or device, and then recapture the image.

Because such conventional imaging techniques require a highly skilled operator, they are often inaccessible outside of a clinical setting. Most untrained operators (e.g., the subjects themselves) are not capable of positioning themselves properly or knowing when to capture images. Furthermore, hiring a skilled operator and/or training an operator is expensive and resource intensive. Therefore, access to such techniques is limited. Consequently, access to the health benefits (e.g., diagnostic, prognostic, surgical assistance, etc.) associated with such techniques is limited.

Some conventional techniques for positioning a subject and imaging device with respect to one another involve using two cameras to capture depth and coordinate information about the position of the target and device. However, such conventional techniques require the use of two cameras, in addition to the imaging device and other existing system optics. The addition of the cameras increases the cost and size associated with the imaging device, contributing to reduced accessibility to such devices outside of the clinical setting and in regions and/or populations with limited resources. Furthermore, it is challenging to find space and optical clearance for the two cameras. First, the space in front the target (e.g., the eye) is typically blocked by an imager lens, which is positioned in close proximity to the eye. Second, when imaging an eye, it is challenging to navigate around human facial structures such as the nose, cheeks, and eyebrows. Human faces vary with respect to structure size and placement, making it challenging to properly place the two cameras so as to enable full visibility of the desired beam path for the full patient population.

Accordingly, the inventors have developed techniques that address the above-described challenges associated with conventional imaging devices. In some embodiments, the techniques developed by the inventors include localizing an imaging target (e.g., a subject's pupil) in a field of view of a first imaging device (e.g., a pupil imaging device) in response to receiving light transmitted through a split image component (e.g., a prism configuration, a mirror configuration, etc.) of the first imaging device. In some embodiments, this includes using the received light to generate an image (e.g., of the subject's pupil), which may be used to localize (e.g., determine a position of) the imaging target with respect to the first imaging device. In some embodiments, results of the localization (e.g., the determined position of the subject's pupil) may be used to adjust an imaging apparatus that includes the first imaging device (e.g., manually, or automatically) and/or to instruct the subject to adjust their position with respect to the imaging apparatus. For example, the techniques may include automatically adjusting the apparatus and/or generating instructions that indicate how to adjust the positioning of the subject and/or apparatus. In some embodiments, the imaging apparatus also includes a second imaging device (e.g., a fundus imaging device). After localizing and repositioning the imaging apparatus and/or imaging target, the second imaging device may be used to obtain an image and/or measurement (e.g., an image and/or measurement of at least a portion of the subject's fundus).

Accordingly, the techniques developed by the inventors are an improvement to the conventional techniques because they enable an unskilled operator (e.g., the subject) to properly position the subject and/or the imaging apparatus to obtain medical grade images and/or support fully automatic image capture. For example, the techniques may be used to automatically adjust the imaging apparatus and/or to output instructions to an operator which indicate how to adjust the subject and/or apparatus. Furthermore, as described herein in more detail, a split image component such as a prism configuration or mirror configuration may be incorporated in the imaging apparatus utilizing existing optical components, avoiding a significant increase in size or expense, and thereby preserving accessibility. For example, the split image component may be incorporated within the same beam path as a fundus imaging device itself. Among other benefits, and in contrast to the dual camera positioning system, incorporating the split image component within the same beam path of the fundus imaging device avoids possible interference issues with different facial structures among the human population.

Following below are descriptions of various concepts related to, and embodiments of, techniques for imaging a target using a split image component such as a prism configuration or a mirror configuration. It should be appreciated that various aspects described herein may be implemented in any of numerous ways, as the techniques are not limited to any particular manner of implementation. Examples of details of implementations are provided herein solely for illustrative purposes. Furthermore, the techniques disclosed herein may be used individually or in any suitable combination, as aspects of the technology described herein are not limited to the use of any particular technique or combination of techniques.

### Exemplary Systems and Methods for Localizing an Imaging Target

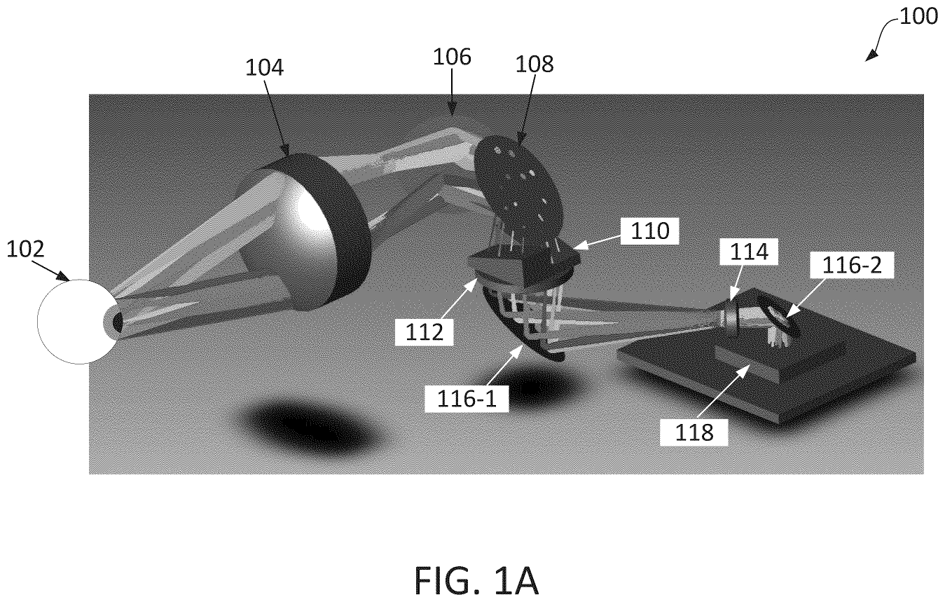

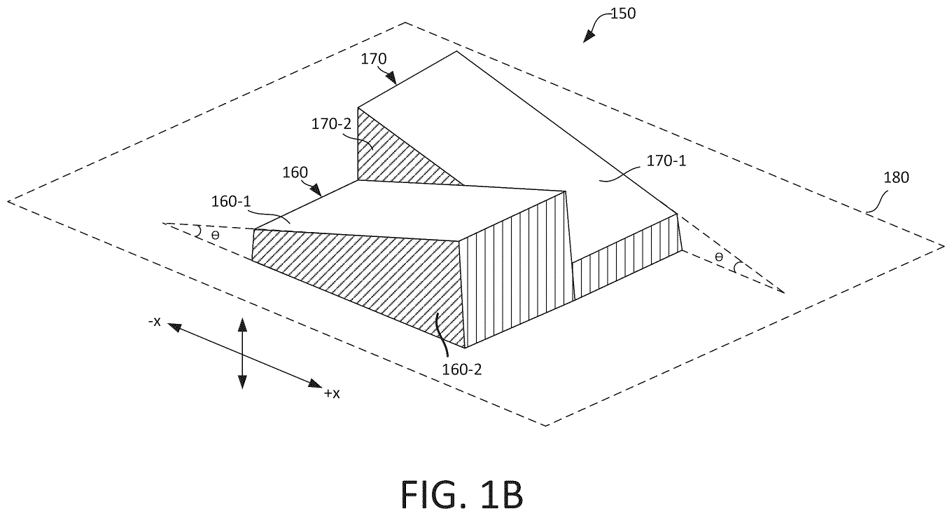

FIG. 1A is a schematic view of exemplary components of an imaging system 100 including a prism configuration, according to some embodiments. As shown, system 100 includes objective lens 104, prism configuration 110, field lens 112, focusing relay lens 114, and image sensor 118. In some embodiments, the system 100 additionally, or alternatively, includes beam splitter 106, beam splitter 108, and fold mirrors 116-1 and 116-2. It should be appreciated that system 100 may include additional or alternative components which are not illustrated in FIG. 1A. For example, the system 100 may include one or more additional fold mirrors, beam splitters, imaging sensors, lenses, or any other suitable component, as aspects of the technology described herein are not limited in this respect.

In some embodiments, one or more of the components of system 100 may be included in an imaging apparatus and form at least a portion of a first imaging device (e.g., a pupil imaging device) included in the apparatus. For example, components along the optical path between the target 102 and the image sensor 118 (e.g., a pupil image sensor) may form at least a part of the first imaging device.

Additionally, or alternatively, in some embodiments, one or more of the components of system 100 may form at least a portion of a second imaging device (e.g., a retinal imaging device) included in the imaging apparatus. For example, components along an optical path between the target 102 and another image sensor (not shown) may form at least a part of the second imaging device.

In some embodiments, the imaging system 100 may be configured to support an optical path between at least a portion of the imaging target 102 (e.g., the pupil) and image sensor 118. In the example of FIG. 1A, objective lens 104 receives light from the imaging target 102. Beam splitter 106 receives light from the objective lens 104 and reflects at least a portion of the light in the direction of beam splitter 108. Beam splitter 108 receives the light reflected from beam splitter 106 and reflects at least a portion of the received light in the direction of prism configuration 110. The light is transmitted through the prism configuration 110 followed by the field lens 112 and is reflected from fold mirror 116-1. The focusing relay lens 114 receives the light reflected from fold mirror 116-1 and transmits the light towards fold mirror 116-2. The light is reflected from fold mirror 116-2 onto image sensor 118. It should be appreciated that the optical path depicted in FIG. 1A is a nonlimiting example, and that any suitable optical path may be supported using any suitable components, as aspects of the technology described herein are not limited in this respect.

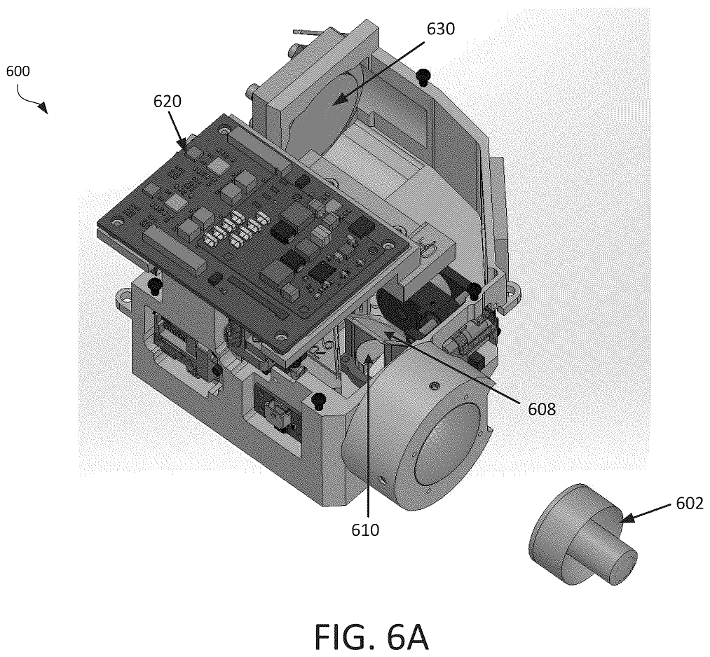

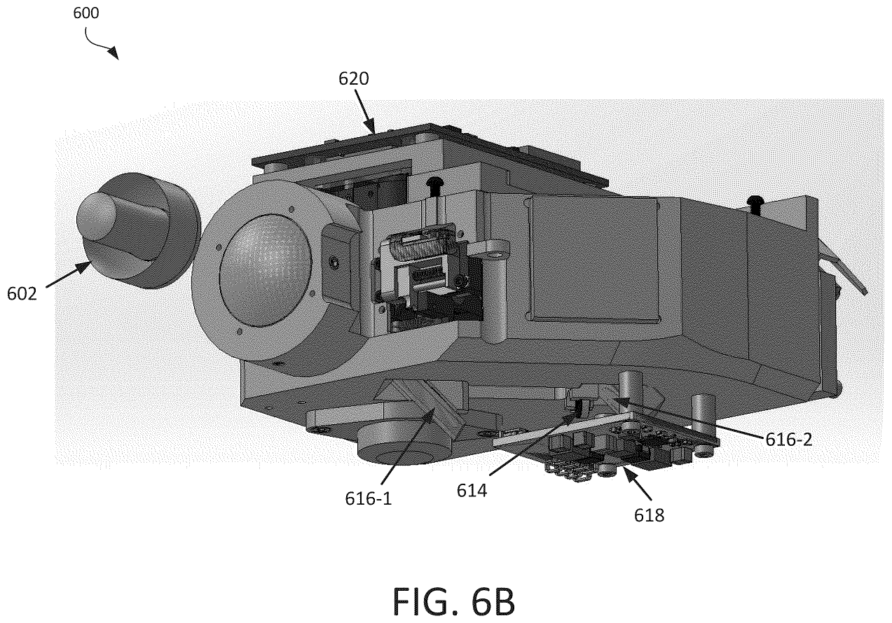

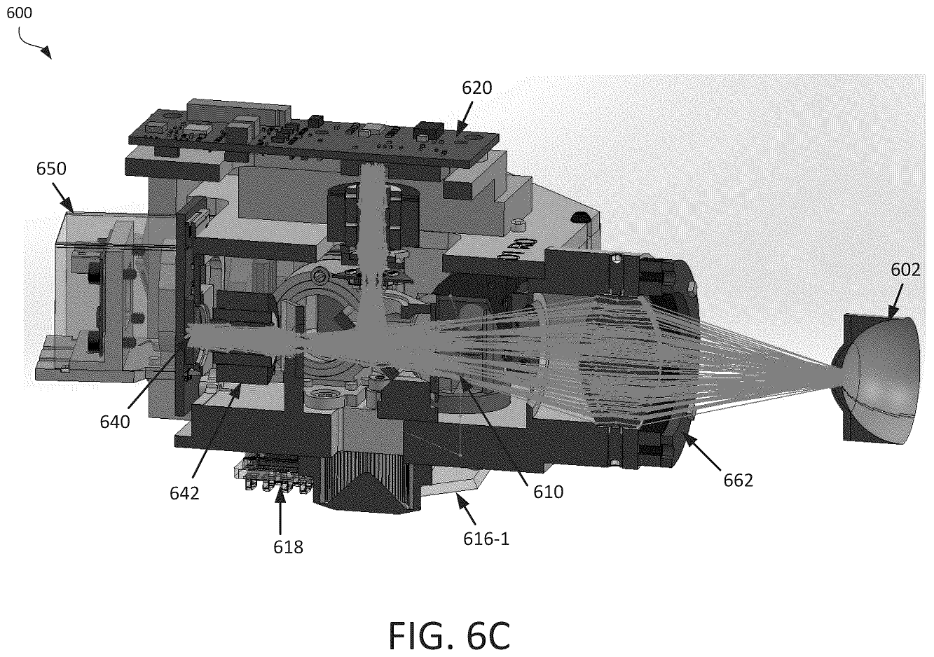

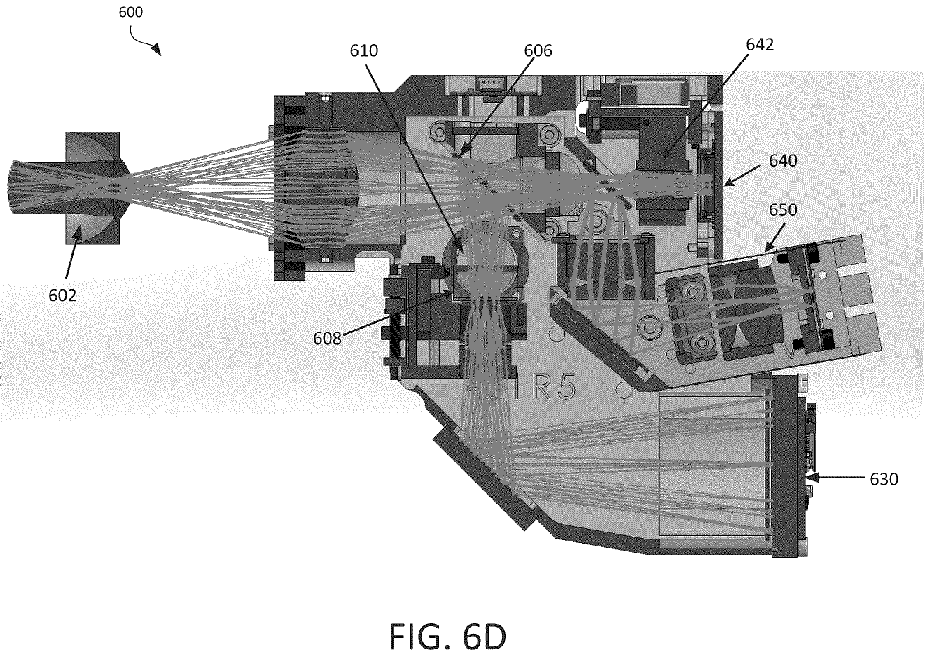

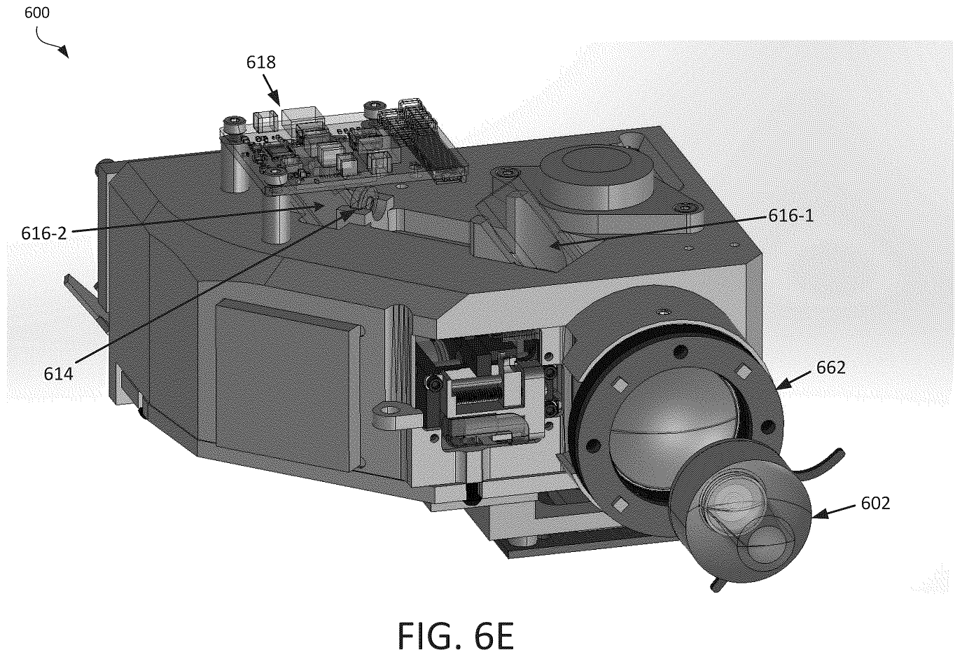

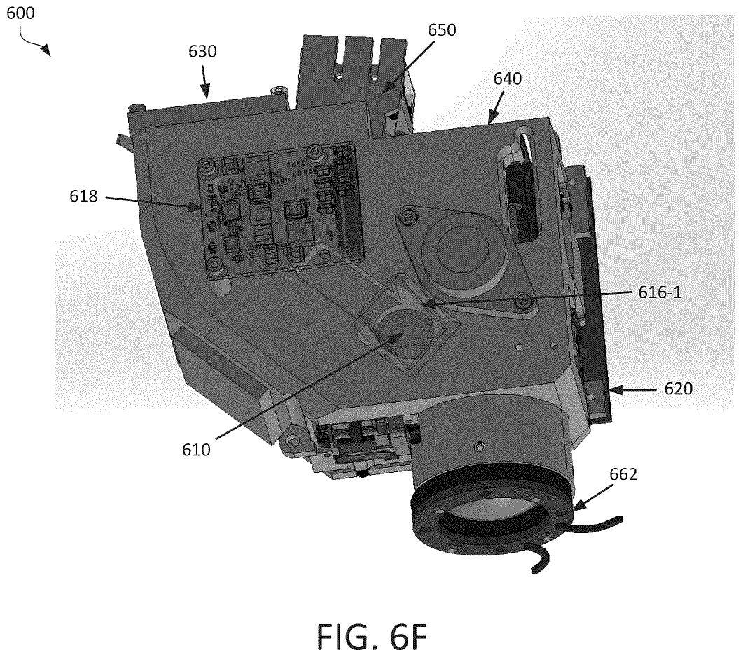

The objective lens 104, in some embodiments, is configured to provide light from an illumination source to the imaging target 102. For example, the imaging system 100 may include source components configured to illuminate the imaging target 102. The source components may include any suitable source components such as, for example, light emitting diodes (LED), infrared (IR) light sources, lasers, and/or any other suitable components that are configured to generate illumination light, as aspects of the technology described herein are not limited in this respect. For example, the imaging system 100 may include (a) one or more IR illuminators (e.g., a ring of IR illuminators) and/or (b) a white light source (e.g., one or more LEDs). FIGS. 6C and 6D show example illumination components 650 and illumination ring 662 of an example imaging apparatus.

In some embodiments, IR illuminators may be used during positioning of the imaging target 102 and/or apparatus, and the white light source may be used during imaging of at least a portion of the target 102 (e.g., during imaging of the fundus). The IR illuminators may be beneficial during localization of the target 102, when the target 102 is an eye; the invisible illumination may allow for localization without causing the pupil to close. This enables a retinal image and/or measurement to be obtained as soon as the target 102 is positioned. By contrast, visible illumination may cause the pupil to close, which may prohibit taking a retinal image as the beam path is too small to effectively image the retina. The reduced pupil size may also lead to image artifacts and/or low image quality.

The objective lens 104, in some embodiments, is additionally or alternatively configured to provide light from a fixation target to the imaging target 102. For example, the imaging system 100 may include fixation target when the imaging target 102 is a subject's eye. The fixation target may be used to guide the subject's eye to a specific position and/or orientation. In some embodiments, a fixation target may include a display (e.g., a liquid crystal display (LCD) panel) or any other suitable components, as aspects of the technology described herein are not limited in this respect. An example fixation target 630 is included in the example imaging apparatus shown in at least FIG. 6D.

The objective lens 104, in some embodiments, provides light received from the imaging target 102 to one or more other optical components of the imaging apparatus. For example, the objective lens 104 may provide light to one or more detection components (e.g., camera(s)). The objective lens may include any suitable objective lens of any suitable dimensions, as aspects of the technology described herein are not limited in this respect.

In some embodiments, beam splitter 106 and beam splitter 108 are each configured to receive light, (a) reflect at least a portion of the received light, and (b) transmit at least a portion of the received light. For example, as shown in FIG. 1A, beam splitter 106 is configured to reflect a portion of the light towards beam splitter 108 and may further be configured to transmit a portion of the light in the direction of another (e.g., a second) optical path (e.g., towards any suitable type of camera, image sensor, etc.). Beam splitter 106 may additionally, or alternatively, be configured to transmit light towards the imaging target 102 (e.g., from an illumination source). Beam splitter 108 is configured to reflect a portion of the light toward prism configuration 110 and may further be configured to transmit a portion of the beam of light in the direction of another (e.g., a third) optical path (e.g., towards any suitable type of camera, image sensor, etc.). Beam splitter 108 may additionally, or alternatively, be configured to transmit light towards the imaging target 102 (e.g., from a fixation target). It should be appreciated that the imaging system 100 may include any suitable number (e.g., 0, 1, 2, 3, etc.) of beam splitters, arranged in any suitable manner, to support any suitable number of optical paths through system 100, as aspects of the technology are not limited in this respect.

In some embodiments, the prism configuration 110 includes a pair of glass wedge prisms. An example split prism configuration is shown in FIG. 1B. As shown in FIG. 1B, the example split prism configuration 150 includes a first prism 160 and a second prism 170. The base of prism 160 and the base of prism 170 are coplanar along plane 180. Additionally, or alternatively, prism 160 and prism 170 are adjacent to one another. For example, side 170-2 of prism 170 is adjacent to the side of prism 160 that is opposite of side 160-2. In some embodiments, the adjacent sides of the prisms 160 and 170 may be in contact with one another. As further shown in FIG. 1B, the top face 160-1 of prism 160 and the top face 170-1 of prism 170 may be positioned at an angle (O) relative to the plane 180. The angle may include any suitable angle, as aspects of the technology described herein are not limited in this respect. In some embodiments, prism 160 is positioned opposite prism 170, such that the top face 160-1 of prism 160 is positioned at an angle θ relative to the positive x-axis, while the top face 170-1 of prism 170 is positioned at an angle of θ relative to the negative x-axis.

In some embodiments, as light passes through the prism configuration 150, a first portion of the light will pass through first prism 160 along a first imaging path and a second portion of the light will pass through the second prism 170 along a second imaging path. The first imaging path and the second imaging path may be separated by an angle.

Referring again to FIG. 1A, in some embodiments, the field lens 112 is a lens or group of lenses configured to redirect light emitted from prism configuration 110 so that only a certain off-axis portion of that light can be passed through the limited aperture of the relay lens 114. In some embodiments, the field lens 112 is adjacent to the prism configuration 110. For example, the field lens 112 may be adjacent to and in contact with the prism configuration 110. Alternatively, the field lens 112 may be adjacent to and separated from the prism configuration 110.

In some embodiments, fold mirror 116-1 and fold mirror 116-2 are included in the imaging system 100 to direct light along a particular path. For example, as shown in FIG. 1A, fold mirror 116-1 is configured to reflect light from field lens 112 to relay lens 114. Fold mirror 116-2 is configured to reflect light from relay lens 114 to image sensor 118.

In some embodiments, the relay lens 114 is a lens or group of lenses that may be used to extend the length of an imaging system and/or invert images. For example, the relay lens 114 may operate by producing intermediate planes of focus, enabling the relay of an image with 1:1 magnification. In some embodiments, the relay lens 114 relays the intermediate image that exists at the prism configuration 110 to image sensor 118 via fold mirror 116-2.

The image sensor 118 may include any suitable image sensor configured to use the received light to generate an image. In some embodiments, the image sensor 118 includes a sensing element such as, for example, a two-dimensional array of pixels. The sensing element may be monochrome or color. The sensing element may be a complementary metal-oxide-semiconductor (CMOS) chip, charge-coupled device (CCD) chip, or any other suitable sensing element, as aspects of the technology described herein are not limited in this respect. As a non-limiting example, the sensing element may be the Sony IMX273 CMOS image sensor with 1440×1080 pixels.

In some embodiments, the image sensor 118 includes a sensing element integrated in an image sensor Printed Circuit Board (PCB). The image sensor PCB may be a custom or commercially available PCB. In some embodiments, the image sensor PCB is configured to output the generated images (e.g., to a different computing device) using an output interface.

In some embodiments, the imaging system 100 may additionally, or alternatively, include one or more computing devices configured to process the image generated using image sensor 118. In some embodiments, the one or more computing devices include the image sensor 118. Additionally, or alternatively, the one or more computing devices may include one or more computing devices separate from the image sensor 118. Such computing device(s) may be included in an imaging apparatus that also includes one or more of the components shown in FIG. 1A. Additionally, or alternatively, the computing device(s) may be located externally from such an imaging apparatus. In some embodiments, the computing device(s) may indirectly receive image data from the image sensor 118 when the computing device(s) are located externally from the imaging apparatus. For example, the computing device(s) may obtain the image data via at least one communication network, such as the Internet or any other suitable communication network(s), as aspects of the technology described herein are not limited in this respect.

In some embodiments, one or more of the components of the imaging system 100 may be incorporated into an imaging apparatus. An example of an imaging apparatus is described herein including at least with respect to FIGS. 6A-6F.

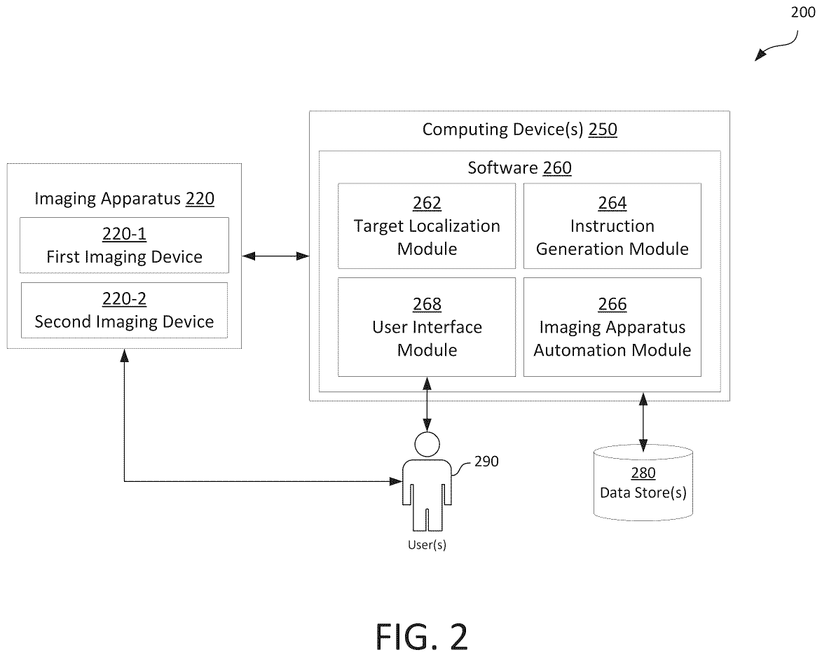

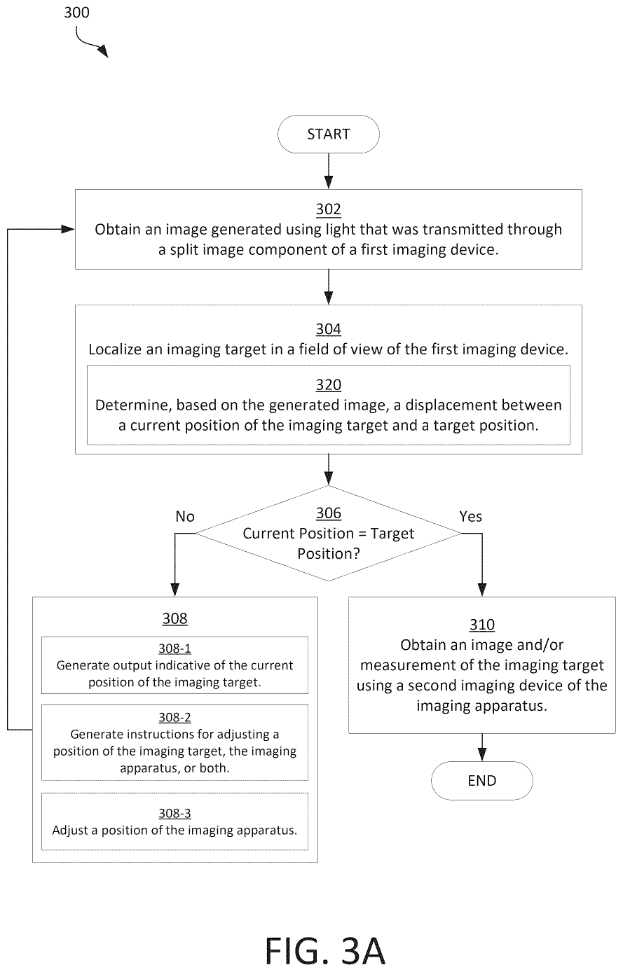

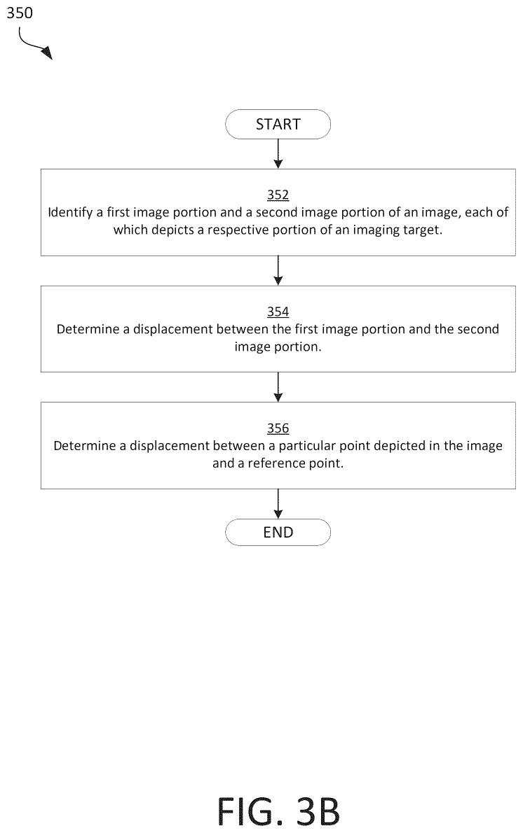

FIG. 2 is a block diagram of an example system 200 for localizing a subject's pupil, according to some embodiments. System 200 includes imaging apparatus 220 and computing device(s) 250. Computing device(s) 250 may be part of or external to imaging apparatus 220. For example, computing device(s) 250 may form part of the first imaging device 220-1, which may be configured to perform one or more (e.g., all) of the acts of process 300 and/or process 350 shown in FIGS. 3A-3B.

Computing device(s) 250 are configured to have software 260 execute thereon to perform various functions in connection with localizing an imaging target. In some embodiments, software 260 includes a plurality of modules. A module may include processor-executable instructions that, when executed by at least one computer hardware processor, cause the at least one computer hardware processor to perform the function(s) of the module. Such module(s) are sometimes referred to herein as “software modules,” each of which includes processor-executable instructions configured to perform one or more processes, such as processes 300 and 350 described herein including at least with respect to FIG. 3A and FIG. 3B, respectively.

The computing device(s) 250 may be operated by one or more user(s) 290. For example, the user(s) 290 may include one or more individuals who are using imaging apparatus 220 (e.g., subjects, clinicians, doctors, untrained operators, researchers, etc.). In some embodiments, the user(s) 290 may provide input specifying processing or other methods to be performed on image data. Additionally, or alternatively, the user(s) 290 may provide input specifying automation instructions for the imaging apparatus. Additionally, or alternatively, user(s) 290 may access results of processing image data. For example, user(s) 290 may access results indicating a position of an imaging target with respect to imaging apparatus 220. Additionally, or alternatively, user(s) 290 may access the image data. Additionally, or alternatively, user(s) 290 may access data indicative of instructions for positioning the target and/or the imaging apparatus 220.

As shown in FIG. 2, software 260 includes multiple software modules for localizing a target (e.g., a pupil), generating output, and/or automating an imaging apparatus (e.g., imaging apparatus 220). Such software modules include a target localization module 262, instruction generation module 264, and imaging apparatus automation module 266.

In some embodiments, the target localization module 262 obtains image data from the imaging apparatus 220, the user(s) 290 (e.g., by the user(s) uploading the image data), and/or the data store(s) 280.

In some embodiments, the target localization module 262 is configured to localize an imaging target (e.g., a pupil) with respect to a target position. The target position may be specified using any suitable coordinates, as aspects of the technology described herein are not limited in this respect. For example, the target position may be specified by a coordinate along an axis (e.g., the z-axis) substantially parallel to a direction in which at least a first imaging device (e.g., a pupil imaging device) is configured to transmit light. Additionally, or alternatively, the target position may be specified by a pair of coordinates in a plane (e.g., the x-y plane) substantially perpendicular to the direction in which the at least one first imaging device is configured to transmit light. Such a coordinate may correspond to the center of the field of view of the first imaging device (e.g., pupil imaging device), for example.

In some embodiments, localizing the imaging target with respect to the target position includes determining a displacement between a current position of the imaging target and the target position. In some embodiments, the target localization module 262 is configured to determine the displacement between the current position and the target position using image data. For example, the image data may include an image generated using light that was transmitted through a split image component such as, for example, a prism configuration (e.g., the prism configuration 110 shown in FIG. 1A) or a mirror configuration.

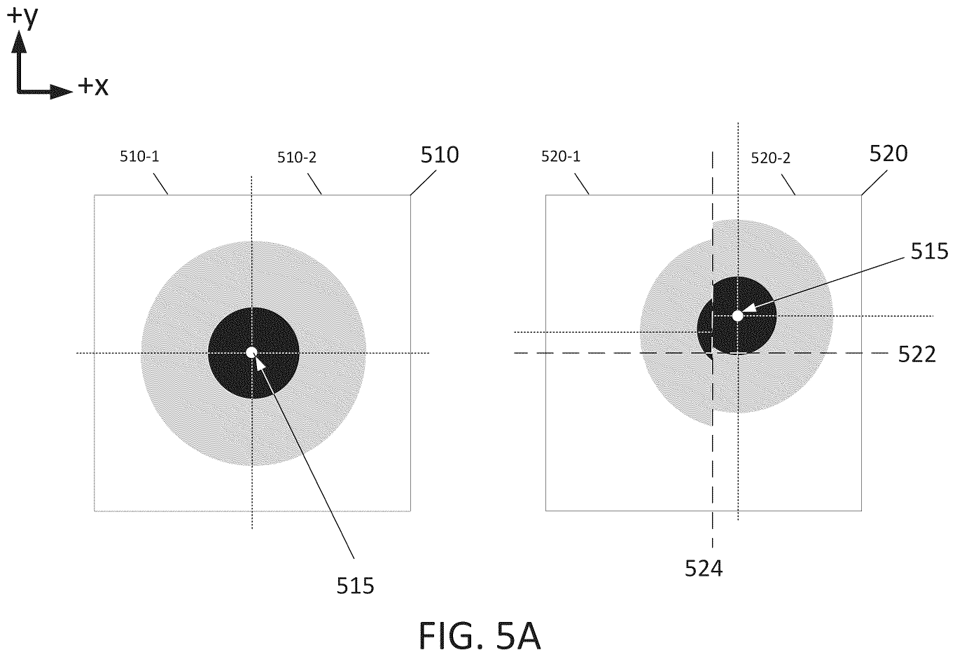

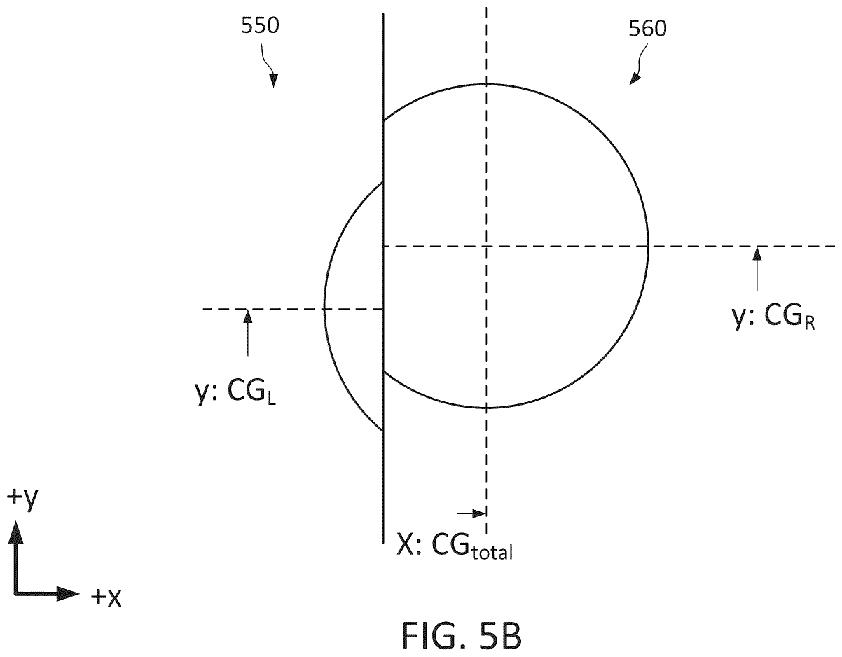

In some embodiments, determining the displacement includes determining a physical displacement between the current position and the target position along an axis substantially parallel to the direction in which the at least a first imaging device (e.g., a pupil imaging device) is configured to transmit light. Such a displacement may indicate that the imaging target is positioned too far or too close to the imaging apparatus 220. In some embodiments, to determine such a displacement, the target localization module 262 may be configured to process image data. The image data may be processed to determine a displacement between at least two portions of the image. For example, each image portion may depict a portion of the imaging target, as shown in the example of FIG. 5A. If the portions of the image are substantially aligned (e.g., image 510 in FIG. 5A), then this may indicate that there is zero physical displacement between the current position and the target position. If the portions of the image are misaligned (e.g., image 520 in FIG. 5A), then the displacement between the image portions may be used to determine the physical displacement between the current position and the target position of the imaging target.

In some embodiments, determining the displacement includes determining a physical displacement between the current position and the target position in a plane substantially perpendicular to the direction in which at least a first imaging device (e.g., a pupil imaging device) is configured to transmit light. Such a displacement may indicate that the imaging target is off center with respect to the field of view of the imaging apparatus 220. In some embodiments, to determine such a displacement, the target localization module 262 may be configured to process the image to determine a displacement between a particular point associated with the imaging target (e.g., the center of the imaging target) and a reference point corresponding to the target position (e.g., the center of the image). For example, in image 520 shown in FIG. 5A, there is a displacement between the center of the pupil 515 and the center of the image 520 along the x-axis 522 and the y-axis 524. In some embodiments, the target localization module 262 may use the displacement(s) to determine the physical displacement between the current and target position of the imaging target.

In some embodiments, the target localization module 262 may be configured to execute one or more acts of process 300 and/or process 350 described herein including at least with respect to FIG. 3A and FIG. 3B, respectively.

In some embodiments, the instruction generation module 264 obtains target localization results from target localization module 262, data store(s) 280, and/or the user(s) 290 (e.g., by the user(s) uploading the target localization data).

In some embodiments, the instruction generation module 264 is configured to generate instructions indicative of the displacement between the current position of the imaging target and the target position. For example, the instruction generation module 264 may be configured to generate instructions to be provided to user(s) 290. The instructions may indicate to user(s) 290 whether to reposition the imaging target, the imaging apparatus 220, or both. Additionally, or alternatively, the instructions may indicate how the target and/or apparatus 220 should be repositioned (e.g., move 1 centimeter forward, etc.) by user(s) 290. Additionally, or alternatively, the instructions may indicate that an image and/or measurement may be obtained (e.g., when there is no displacement between the target position and the current position or when the displacement between the target position and the current position is less than a threshold displacement). In some embodiments, the instruction generation module 264 may additionally or alternatively be configured to generate instructions to be provided to the imaging apparatus automation module 266. For example, the instructions may indicate how the imaging apparatus 220 should be repositioned, whether an image and/or measurement may be obtained, or any other suitable instructions, as aspects of the technology described herein are not limited in this respect.

In some embodiments, the imaging apparatus automation module 266 (“automation module 266”) is configured to obtain instructions from instruction generation module 264, data store(s) 280, and/or user(s) 290 (e.g., by the user(s) uploading the instructions).

In some embodiments, the automation module 266 is configured to transmit instructions to the imaging apparatus 220. The instructions may be configured to cause the imaging apparatus to perform any suitable function, as aspects of the technology described herein are not limited in this respect. For example, the instructions may be configured to cause the imaging apparatus to automatically change a position of the imaging apparatus 220 relative to an imaging target. Additionally, or alternatively, the instructions may be configured to cause the imaging apparatus 220 to obtain an image and/or measurement of the imaging target. In some embodiments, the automation module 266 may communicate the instructions via at least one wired or wireless communication network, such as the Internet or any other suitable communication network(s), as aspects of the technology described herein are not limited in this respect.

In some embodiments, software 260 further includes user interface module 268. User interface module 268 may be configured to generate a graphical user interface through which a user may provide input and view information generated by software 260. For example, in some embodiments, the user interface module 268 may be a webpage or web application accessible through an Internet browser. In some embodiments, the user interface module 268 may generate a graphical user interface (GUI) of an app executing on the user's mobile device. In some embodiments, the user interface module 268 may generate a GUI on an imaging apparatus such as the imaging apparatus 220. In some embodiments, the user interface module 268 may generate a number of selectable elements through which a user may interact. For example, the user interface module 268 may generate dropdown lists, checkboxes, text fields, or any other suitable element.

In some embodiments, the user interface module 268 is configured to generate a GUI including any suitable information, as aspects of the technology described herein are not limited in this respect. For example, the user interface module 268 may be configured to generate a GUI including image data generated using an optical path having a split image component (e.g., image data used for localizing the imaging target). Additionally, or alternatively the user interface module 268 may be configured to generate a GUI including results of localizing the imaging target. For example, the GUI may indicate displacement of image portions with respect to one another and/or displacement between two positions in the image. Additionally, or alternatively, the user interface module 268 may be configured to generate a GUI indicating displacement between the current position of the imaging target and the target position. Additionally, or alternatively, the user interface module 268 may be configured to generate a GUI including instructions generated by instruction generation module 264.

As shown in FIG. 2, system 200 also includes imaging apparatus 220. The imaging apparatus 220 may include any suitable imaging apparatus that includes at least: (a) a first imaging device 220-1 configured to obtain an image of at least a first portion of an imaging target (e.g., a pupil), (b) a second imaging device 220-2 configured to obtain an image of at least a second portion of an imaging target (e.g., a fundus), and (c) a split image component, such as a prism configuration or mirror configuration, which is positioned along an optical path between the imaging target and at least the first imaging device 220-1. For example, the imaging apparatus 220 may include the imaging system 100 shown in FIG. 1A and/or the example imaging apparatus 600 shown in FIGS. 6A-6F.

In some embodiments, user(s) 290 may operate (e.g., provide input to) the imaging apparatus 220. The user(s) 290 may include one or more of the user(s) that operate the computing device(s) 250. Alternatively, the user(s) 290 may differ from those that operate the computing device(s) 250.

System 200 further includes data store(s) 280. In some embodiments, the data store(s) 280 stores image and/or measurement data obtained from imaging apparatus 220. Additionally, or alternatively, the data store(s) 280 may store results of localizing the imaging target (e.g., using target localization module 262). Additionally, or alternatively, the data store(s) 280 may store instructions generated by the instruction generation module 264 or obtained from user(s) 290. Additionally, or alternatively, the data store(s) 280 may store data about target positions associated with different imaging targets. It should be appreciated that the data store(s) 280 may store any other suitable type of information, as aspects of the technology described herein are not limited in this respect.

The data store(s) 280 may be of any suitable type (e.g., database system, multi-file, flat file, etc.) and may store data in any suitable way in any suitable format, as aspects of the technology described herein are not limited in this respect. The data store(s) 280 may be part of or external to the computing device(s) 250.

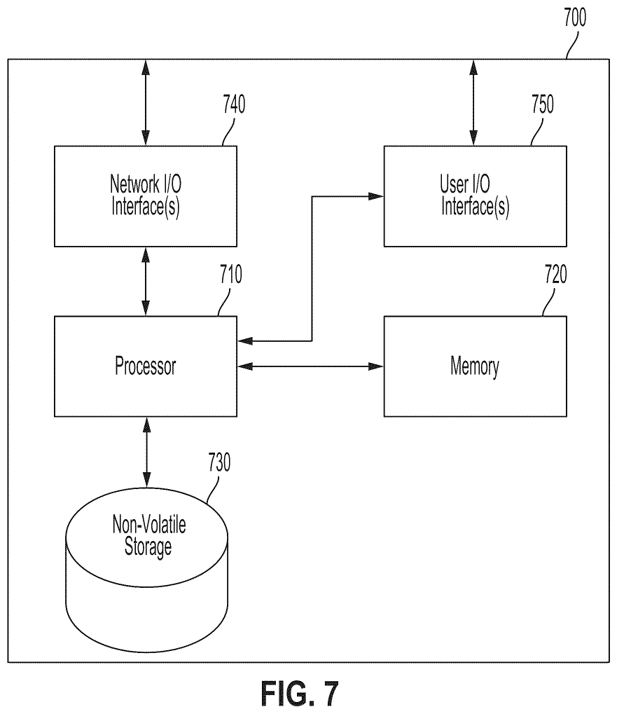

FIG. 3A is a flowchart of an illustrative process for localizing a subject's pupil, according to some embodiments. One or more of acts (e.g., all of the acts) of process 300 may be performed automatically by any suitable computing device(s). For example, the act(s) may be performed by a System on Module (SOM) computer, laptop computer, a desktop computer, one or more servers, in a cloud computing environment, computing device(s) 250 described herein with respect to FIG. 2, computing device 700 described herein with respect to FIG. 7, and/or in any other suitable way.

At act 302, an image is obtained. In some embodiments, the image was previously-generated using light that was transmitted through a split image component (e.g., a prism configuration, a mirror configuration, etc.) of a first imaging device of an imaging apparatus. In some embodiments, the image depicts at least a portion of an imaging target. For example, the image may depict at least a portion of a subject's pupil.

In some embodiments, the first imaging device may be an imaging device configured to obtain an image of a pupil or any other suitable imaging target, as aspects of the technology described herein are not limited in this respect. In some embodiments, the image may be obtained from an image sensor of the first imaging device (e.g., image sensor 118 shown in FIG. 1A). Additionally, or alternatively, the image may be obtained from a data store, from a user (e.g., by uploading the image), or in any other suitable manner, as aspects of the technology described herein are not limited in this respect.

At act 304, the imaging target is localized in a field of view of the first imaging device. In some embodiments, this includes determining, at act 320, a displacement between a current position of the imaging target and a target position of the imaging target.

In some embodiments, determining the displacement may include determining a displacement within a plane that is substantially parallel to the direction in which the first imaging device is configured to transmit light. For example, this may include determining whether the imaging target is positioned too far to the left, too far to the right, too high, or too low with respect to the field of view of the first imaging device. In some embodiments, determining such a displacement includes processing the image obtained at act 302. For example, this may include (a) identifying a point in the image depicting a point on the imaging target (e.g., the center of the pupil), and (b) determining a displacement between the point and a reference point (e.g., the center of the image). Example techniques for determining such a displacement are described herein including at least with respect to FIG. 3B, FIGS. 5A-5B.

In some embodiments, determining the displacement may additionally, or alternatively, include determining a displacement along an axis that is substantially perpendicular to the direction in which the first imaging device is configured to transmit light. For example, this may include determining whether the imaging target is too close to or too far away from the first imaging device. In some embodiments, determining such a displacement includes processing the image obtained at act 302. As a result of having been generated using light that was transmitted through a split image component, the obtained image may include at least two image portions. Each image portion may depict a corresponding portion of the imaging target. If the imaging target is positioned at the target position, then the portions of the imaging target depicted in the image will be substantially aligned with one another (e.g., example image 510 shown in FIG. 5A). If the imaging target is not positioned at the target position (e.g., too far from or too close to the first imaging device), then the portions of the imaging target depicted in the image will be misaligned (e.g., example image 520 shown in FIG. 5A). Accordingly, in some embodiments, determining the displacement between the current position and the target position may include determining a degree to which the image portions are misaligned. Example techniques for determining such a displacement are described herein including at least with respect to FIG. 3B, FIG. 5A and FIG. 5B.

At act 306, process 300 includes determining whether the current position of the imaging target is substantially equal to the target position. For example, if there is no displacement (or below a threshold displacement) between the current and target position, as determined at act 304, then the current position is determined to be substantially equal to the target position. By contrast, if there is a displacement between the current and target position, as determined at act 304, then the current position is determined to not equal the target position.

If the current position of the imaging target is determined to not equal the target position, at act 306, then process 300 proceeds to act 308. Act 308 may include one or more of sub-act 308-1, sub-act 308-2, and sub-act 308-3.

At sub-act 308-1, output (e.g., feedback) indicative of the displacement between the current position and the target position of the imaging target is generated. For example, the output may indicate the displacement between the current position and the target position. In some embodiments, the output is generated using a user interface module (e.g., user interface module 268 shown in FIG. 2) configured to generate such an output. In some embodiments, the output is provided in the form of a report displayed via a graphical user interface (GUI) or in any other suitable manner, as aspects of the technology described herein are not limited in this respect.

At sub-act 308-2, instructions for adjusting the position of the imaging target, the imaging apparatus, or both, are generated. In some embodiments, the instructions may be generated using an instruction generation module (e.g., instruction generation module 264 shown in FIG. 2) configured to generate such instructions. In some embodiments, the instructions indicate how the imaging target should be positioned. For example, the instructions may indicate a distance and/or direction in which to move the target and/or imaging apparatus. Such instructions may be provided to an operator (e.g., the subject being imaged and/or measured or another operator) of the imaging apparatus, who may use the instructions to position the target and/or the apparatus. Additionally, or alternatively, the operator may convey the instructions to a subject being imaged and/or measured, who may then position themselves according to the instruction.

In some embodiments, the instructions may be output to the user in any suitable format, as aspects of the technology described herein are not limited in this respect. For example, the instructions may include written instructions for positioning the target and/or apparatus. Additionally, or alternatively, the instructions may be provided using visual and/or audio aids that indicate where the imaging target should be positioned. In some embodiments, the output is generated using a user interface module (e.g., user interface module 268 shown in FIG. 2) configured to generate such an output. In some embodiments, the output is provided in the form of a report displayed via a graphical user interface (GUI) or in any other suitable manner, as aspects of the technology described herein are not limited in this respect.

At sub-act 308-3, the position of the imaging apparatus may be adjusted. In some embodiments, this includes transmitting instructions to the imaging apparatus to cause the imaging apparatus to automatically adjust its position. For example, an automation module (e.g., imaging apparatus automation module 266 shown in FIG. 2) may be configured to transmit instructions to an imaging apparatus to cause the imaging apparatus to perform one or more functions including automatically adjusting its position. The imaging apparatus may include one or more motorized components (e.g., motors and/or actuators) and/or robotic components that enable its automation. Additionally, or alternatively, the position of the imaging apparatus may be adjusted manually by one or more operators (e.g., a subject being imaged and/or measured).

Following act 308, one or more acts of process 300 may be repeated. For example, one or more acts of process 300 may be repeated after the target and/or apparatus are repositioned to determine whether the updated position(s) are sufficient for obtaining an image and/or measurement of the target.

If, at act 306, the current position is determined to equal the target position of the imaging target, then process 300 proceeds to act 310. At act 310, an image and/or measurement is obtained of at least a portion of the imaging target (e.g., the fundus). In some embodiments, this includes transmitting instructions to the imaging apparatus to cause the imaging apparatus to obtain the image and/or measurement of the target. For example, an automation module (e.g., imaging apparatus automation module 266 shown in FIG. 2) may be configured to transmit instructions to an imaging apparatus to cause the imaging apparatus to perform one or more functions including automatically obtaining an image and/or measurement. Additionally, or alternatively, the image and/or measurement may be obtained as a result of one or more operators (e.g., a subject being imaged and/or measured) operating the imaging apparatus. For example, the imaging and/or measurement may be obtained using a second imaging device (e.g., a fundus imaging device) of the imaging apparatus.

The imaging techniques performed at act 310 (e.g., by the second imaging device) may include any suitable imaging techniques as aspects of the technology described herein are not limited in this respect. For example, the imaging techniques may include one or more of fundus imaging, optical imaging, optical coherence tomography (OCT), fluorescence imaging, color optical imaging, infrared (IR) imaging, and/or electro-optical imaging.

It should be appreciated that process 300 may include one or more additional or alternative acts not shown in FIG. 3A. For example, process 300 may include an act for pre-processing the image obtained at act 302 (e.g., thresholding the image). Additionally, or alternatively, process 300 may include an act for storing results of localizing the imaging target at act 304, output generated at act 308-1, and/or instructions generated at act 308-2. For example, the results, output, and/or instructions may be stored in a data store (e.g., data store(s) 280 shown in FIG. 2).

FIG. 3B is a flowchart of an illustrative process for determining a displacement between a current position of at least a portion of the imaging target (e.g., the subject's pupil) and a target position, according to some embodiments. One or more of acts (e.g., all of the acts) of process 350 may be performed automatically by any suitable computing device(s). For example, the act(s) may be performed by a System on Module (SOM) computer, a laptop computer, a desktop computer, one or more servers, in a cloud computing environment, computing device(s) 250 described herein with respect to FIG. 2, computing device 700 described herein with respect to FIG. 7, and/or in any other suitable way.

At act 352, at least two portions of an image (e.g., an image obtained at act 302 of process 300 shown in FIG. 3A) are identified. For example, the image portions may include a first image portion and a second image portion. In some embodiments, each image portion depicts a respective portion of an imaging target (e.g., a pupil). In some embodiments, the image portions result from the image having been generated using light that was transmitted through a split image component of an imaging device (e.g., a pupil imaging device). For example, the image may be a split image, and each of the image portions may correspond to respective half of the split image.

At act 354, a displacement is determined between the first image portion and the second image portion. In some embodiments, this includes determining a displacement between the portion of the imaging target depicted in the first image portion and the portion of the imaging target depicted in the second image portion. For example, this may include (a) determining a first displacement, along a particular axis, of the first image portion relative to a reference position, (b) determining a second displacement, along the particular axis, of the second image portion relative to the reference position, and (c) determining the difference between the first displacement and the second displacement. For example, the reference position may be the center of the image. Example techniques for determining a displacement between image portions are described herein including at least with respect to FIG. 5A and FIG. 5B.

In some embodiments, determining the displacement further includes determining a direction of the displacement between the image portions. The direction of displacement may be informative as to whether the imaging target is too close or too far. For example, if the image is split into a left image portion and a right image portion, and the image depicted in the left image portion appears higher than the right image portion, this may indicate that imaging target is too far from the imaging apparatus. Alternatively, if the image depicted in the left image portion appears higher than the right image portion, this may indicate that the imaging target is too close to the imaging apparatus.

Examples of determining a displacement between a first image portion and a second image portion are described herein including at least with respect to FIG. 5A and FIG. 5B.

In some embodiments, the displacement determined at act 354 may be used to determine whether and how to adjust the positioning of the imaging apparatus and/or the imaging target. In some embodiments, if the determined displacement is less than or equal to a threshold, then the image portions may be considered substantially aligned, and no repositioning may be needed. The threshold may include any suitable threshold (e.g., 0 or greater than 0), as aspects of the technology described herein are not limited in this respect. In some embodiments, if the displacement is above the threshold, then the image portions may be considered misaligned. The displacement and/or the direction of displacement may be used to reposition the imaging apparatus and/or the imaging target. For example, the displacement and direction of displacement may be used to determine how far and in what direction the target and/or apparatus should be moved.

At act 356, a displacement between a particular point depicted in the image and a reference point is determined. For example, the reference point may represent the center of the image and the particular point may represent the point on the imaging target that is to be centered for imaging and/or measurement (e.g., the center of the pupil). In some embodiments, results of act 354 (e.g., displacement of the image portions along an axis) may be used to determine the displacement at act 356.

Examples of determining a displacement between a point depicted in an image and a reference point are described herein including at least with respect to FIG. 5A and FIG. 5B.

In some embodiments, the displacement determined at act 356 may be used to determine whether and how to adjust the positioning of the imaging apparatus and/or the imaging target. In some embodiments, if the determined displacement is less than or equal to a threshold, then the particular point depicted in the image may be considered substantially aligned with the reference point, and no repositioning may be needed. The threshold may include any suitable threshold (e.g., 0 or greater than 0), as aspects of the technology described herein are not limited in this respect. In some embodiments, if the displacement is above the threshold, then the particular point and the reference point may be considered to be misaligned. The displacement may be used to reposition the imaging apparatus and/or the imaging target (e.g., to decrease the displacement between the imaging apparatus and imaging target). For example, the displacement may be used to determine how far and in what direction the target and/or apparatus should be moved.

Though act 352 and act 354 are shown as preceding act 356 in process 350, it should be appreciated that act 356 may be performed contemporaneously with or prior to act 352 and/or act 354, as aspects of the technology described herein are not limited in this respect.

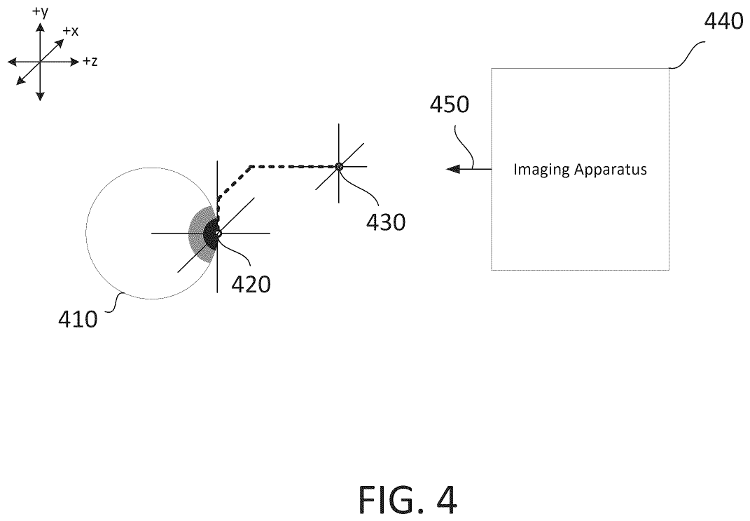

FIG. 4 shows an example of displacement between a current position and a target position of an imaging target, as aspects of the technology described herein are not limited in this respect. As shown in FIG. 4, the imaging target is the pupil of an eye 410. The pupil is positioned at a current position 420, which differs from the target position 430.

In some embodiments, a displacement between the current position 420 and the target position may be determined using at least some of the techniques described herein including at least with respect to FIG. 3A and FIG. 3B. This may include determining a displacement along the z-axis, which is substantially parallel to the direction, indicated by arrow 450, in which the imaging apparatus 440 is configured to transmit light to the subject's eye 410. This may additionally or alternatively include determining a displacement within the x-y plane, which is substantially perpendicular to the direction in which the imaging apparatus 440 is configured to transmit light.

In some embodiments, after determining the displacement between the current position 420 and the target position 430, the position of the eye 410 and/or a position of the imaging apparatus 440 may be adjusted. In some embodiments, adjusting the position of the eye 410 may involve repositioning the eye 410, and more specifically the pupil, to the target position 430. For example, instructions may be provided to the subject, and the subject may reposition their eye 410 to the target position 430. Additionally, or alternatively, adjusting the position of the imaging apparatus 440 may involve repositioning the imaging apparatus 440 such that the target position 430 is effectively repositioned to the current position 420 of the pupil. For example, instructions may be provided to an operator of the imaging apparatus 440, and the operator may adjust the position of the imaging apparatus 440. Additionally, or alternatively, the imaging apparatus may automatically adjust its positioning in response to a determination of the displacement between the current position 420 and the target position 430.

FIG. 5A shows example images 510 and 520, each of which was generated using light that has been transmitted through a split image component (e.g., a prism configuration, a mirror configuration, etc.) of a first imaging device of an imaging apparatus. Each of the images depicts at least a portion of an eye including the pupil.

In example image 510, the center of the pupil 515 is centered within the image. This indicates that the pupil is correctly positioned within the plane perpendicular to the direction in which the first imaging device is configured to transmit light. In other words, the pupil is not too far to the left, right, up, or down with respect to the center of the image 510. Additionally, in example image 510, there is no displacement between the first image portion 510-1 and the second image portion 510-2 (i.e., the image portions are substantially aligned. This indicates that the pupil is correctly positioned along the axis substantially parallel to the direction in which the first imaging device is configured to transmit light. In other words, the pupil is not too far or too close to the first imaging device.

By contrast, in image 520, the center of the pupil 515 is not centered within the image. Rather, the center of the pupil 515 is displaced along both the x-axis 522 and the y-axis 524 relative to the center of the image 520. This indicates that the center of the pupil 515 is incorrectly positioned within the plane perpendicular to the direction in which the first imaging device is configured to transmit light. In other words, the pupil is positioned too far to the right and too far up with respect to the center of the image 520. Additionally, in example image 510, there is a displacement between the first image portion 510-1 and the second image portion 510-2 (i.e., the image portions are misaligned). This indicates that the pupil is incorrectly positioned along the axis substantially parallel to the direction in which the first imaging device is configured to transmit light. In other words, the pupil is too far or too close to the first imaging device.

FIG. 5B shows an example of determining a displacement between a current position and a target position of an imaging target by processing an image generated using a split image component (e.g., a prism configuration, a mirror configuration, etc.).

In some embodiments, the image may be processed to determine a displacement between the current position and the target position along three axes. For example, with reference to FIG. 4, the image may be processed to determine a displacement between the current position 420 and the target position 430, along the x-axis, the y-axis, and the z-axis.

In some embodiments, processing the image includes one or more image pre-processing steps. For example, pre-processing the image may include thresholding the image so that only the dark pixels (e.g., those within the pupil) are considered.

In some embodiments, processing the image additionally or alternatively includes determining the center of gravity of at least some of the pixels. For example, this may include (a) determining the center of gravity (CG_{L}) of pixels in the y-direction for the image portion 550 (CG_{L}), (b) determining the center of gravity (CG_{R}) of pixels in the y-direction for the image portion 560, and (c) determining the center of gravity (CGₜₒₜₐₗ) in the x-direction for the entire image.

In some embodiments, the determined centers of gravity (CG_{L}, CG_{R}, and CGₜₒₜₐₗ) may be used to determine the displacement between the current position and the target position along the x-axis, y-axis, and z-axis (e.g., as shown in FIG. 4). For example, the displacement between the current position and the target position along the x-axis (Δx), the displacement between the current position and the target position along the y-axis (Δy), and the displacement between the current position and the target position along the z-axis (Δz) may be calculated using the equations shown in Table 1.

TABLE 1
Equations for determining displacement between
a current position and a target position.

| Direction of | |
|---|---|
| Displacement | Equation |
| Δx | x: CGₜₒₜₐₗ |
| Δy | (y: CG_{R} + y: CG_{L})/2 |
| Δz | k(y: CG_{R} − y: CG_{L}), where k is a dimensionless |
| | constant specific to the particular embodiment, whose |
| | magnitude is typically of the order of the reciprocal of |
| | the wedge angle (Θ) in FIG. 1B, expressed in radians |

### Example Applications

The techniques described herein may be applied in many different circumstances for localizing a target, as the techniques are not limited to any particular application. For example, the techniques may be used to localize a pupil in the field of view of a pupil imaging device and, after localizing the pupil, to image and/or measure at least a portion of the eye (e.g., the fundus, the retina, etc.) using a second imaging device (e.g., a fundus imaging device). Localizing the pupil may help with positioning the eye and/or the imaging apparatus for obtaining clinically useful images and/or measurements of the eye.

Additionally, or alternatively, the techniques may be applied during surgery to localize a surgical target. For example, a system (e.g., system 100) that includes a split image component may be used to generate an image of a surgical target, and the image may be used to localize the surgical target within the field of view of the system. Localizing the surgical target may help with positioning surgical tools for performing precise and accurate incisions and/or other operations. For example, the target localization system may be included in a robotic system, and the robotic system may adjust its positioning with respect to the surgical target based on results of localizing the surgical target (e.g., based on the displacement determined between a current and target position).

Additionally, or alternatively, the techniques may be applied to photography. For example, a system (e.g., system 100) that includes a split image component may be used to generate an image of a subject, and the image may be used to localize the subject within the field of view of the system. Localizing the subject may help with positioning the camera for capturing an image (e.g., of the fundus) that is in focus and/or centered.

While multiple examples are described above, it should be appreciated that the techniques described herein may be applied to any suitable application, as aspects of the technology described herein are not limited in this respect.

Exemplary Imaging Apparatus with Prism Configuration

FIGS. 6A-6F illustrate an exemplary embodiment of an imaging apparatus 600 having a prism configuration. For example, apparatus 600 may include system 100 described herein with respect to FIG. 1A. It should be appreciated that the imaging apparatus 600 is not limited to the embodiment shown in FIGS. 6A-6F and may include one or more additional or alternative components, as aspects of the technology described herein are not limited to any particular embodiment.

FIG. 6A is a top perspective view of an exemplary imaging apparatus 600 having a prism configuration, according to some embodiments. As shown in FIG. 6A, the imaging apparatus 600 includes fixation target 630, beam splitter 608, prism configuration 610, and camera 620.

In some embodiments, the prism configuration 610 is configured to receive light reflected from a subject's eye 602 and transmit the light to an imaging sensor, such as image sensor 618 shown in FIG. 6B. The prism configuration 610 may be formed of one or more prisms. For example, the prism configuration 610 may be formed of two prisms, such as the two prisms of the prism configuration 110 shown in FIG. 1A and prism configuration 150 shown in FIG. 1B.

In some embodiments, beam splitter 608 may be configured to transmit light from a fixation target, such as fixation target 630, towards the subject's eye 602. Additionally, or alternatively, beam splitter 608 may be configured to reflect light received from the subject's eye towards prism configuration 610.

In some embodiments, fixation target 630 may be configured to display a visible fixation target in various positions to cause the subject's eye to move in particular directions when the subject (e.g., by tracking the fixation target).

In some embodiments, the camera 620 may be configured to receive light from the subject's eye 602 and generate an image in response to receiving the light. The camera may be configured for a particular type or types of imaging. For example, the camera 620 may be configured for autofluorescence imaging or any other suitable type of imaging, as aspects of the technology described herein are not limited in this respect.

FIG. 6B is a front perspective view of the exemplary imaging apparatus of FIG. 6A, according to some embodiments. As shown in FIG. 6B, imaging apparatus 600 further includes mirror 616-1, mirror 616-2, relay lens 614, and image sensor 618.

In some embodiments, mirror 616-1 is configured to direct light received from the prism configuration 610 towards relay lens 614. In some embodiments, mirror 616-1 may receive light directly or indirectly from the prism configuration 610. For example, light that has passed through the prism configuration 610 may travel along an optical path comprising one or more lenses or mirrors prior to arriving at mirror 616-1. Mirror 616-1 may include mirror 116-1 shown in FIG. 1A.

In some embodiments, relay lens 614 is a lens or group of lenses that may be used to relay the intermediate image that exists at the prisms, to the image sensor 618 via fold mirror 616-2.

In some embodiments, light that passes through relay lens 614 is directed towards mirror 616-2. Mirror 616-2 may be configured to reflect the light towards image sensor 618. Mirror 616-2 may include mirror 116-2 shown in FIG. 1A.

Image sensor 618 may include any suitable image sensor configured to use the received light to generate an image. In some embodiments, the image sensor 618 includes a sensing element such as, for example, a two-dimensional array of pixels. In some embodiments, the sensing element is integrated in an image sensor Printed Circuit Board (PCB). The image sensor 618 may include image sensor 118 shown in FIG. 1A.

FIG. 6C is a side cross-sectional view of the exemplary imaging apparatus of FIG. 6A, according to some embodiments. As shown in FIG. 6C, imaging apparatus 600 further includes illumination components 650, illumination components 662, camera 640, and lens assembly 642.

In some embodiments, the illumination components 650 include at least one illumination source used to illuminate the eye 602 for imaging and/or measurement. For example, the illumination source may include at least one white light source (e.g., LEDs), or any other suitable illumination source, as aspects of the technology described herein are not limited in this respect.

In some embodiments, the illumination components 662 may include at least one illumination source for positioning, imaging, and/or measuring the eye 602. For example, the illumination source may include at least one infrared (IR) illumination source (e.g., a ring of IR illuminators) or any other suitable illumination source, as aspects of the technology described herein are not limited in this respect.

In some embodiments, the camera 640 may be configured to receive light from the subject's eye 602 and generate an image in response to receiving the light. The camera may be configured for a particular type or types of imaging. For example, the camera 620 may be configured for fundus imaging or any other suitable type of imaging, as aspects of the technology described herein are not limited in this respect.

In some embodiments, lens assembly 642 may be configured for focusing light from the eye 602, before the light reaches camera 640. The lens assembly 642 may include an MV lens assembly.

FIG. 6D is a top cross-sectional view of the exemplary imaging apparatus of FIG. 6A, according to some embodiments. As shown in FIG. 6C, imaging apparatus 600 further includes beam splitter 606. In some embodiments, the beam splitter 606 is configured to (a) transmit illumination light from illumination components 650 to the eye 602, (b) transmit light from the eye 602 to detection components such as camera 640, and (c) reflect light received from the eye towards beam splitter 608.

FIG. 6E is a front perspective view of the exemplary imaging apparatus of FIG. 6A, according to some embodiments.

FIG. 6F is a bottom perspective view of the exemplary imaging apparatus of FIG. 6A, according to some embodiments.

### Computer Implementation

An illustrative implementation of a computer system 700 that may be used in connection with any of the embodiments of the technology described herein (e.g., such as the processes of FIG. 3A and FIG. 3B) is shown in FIG. 7. The computer system 700 includes one or more processors 710 and one or more articles of manufacture that comprise non-transitory computer-readable storage media (e.g., memory 720 and one or more non-volatile storage media 730). The processor 710 may control writing data to and reading data from the memory 720 and the non-volatile storage device 730 in any suitable manner, as the aspects of the technology described herein are not limited to any particular techniques for writing or reading data. To perform any of the functionality described herein, the processor 710 may execute one or more processor-executable instructions stored in one or more non-transitory computer-readable storage media (e.g., the memory 720), which may serve as non-transitory computer-readable storage media storing processor-executable instructions for execution by the processor 710.

Computing device 700 may include a network input/output (I/O) interface 740 via which the computing device may communicate with other computing devices. Such computing devices may be interconnected by one or more networks in any suitable form, including a local area network or a wide area network, such as an enterprise network, an intelligent network (IN) or the Internet. Such networks may be based on any suitable technology and may operate according to any suitable protocol and may include wireless networks, wired networks or fiber optic networks.

Computing device 700 may also include one or more user I/O interfaces 750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a System on Module (SOM) computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

### CONCLUSION

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The acts performed as part of the methods may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. A method, comprising:
localizing a pupil of a subject's eye in a field of view of a pupil imaging device using an image of at least a portion of the pupil, wherein the image was generated using light that was transmitted from the pupil through a split image component of the pupil imaging device.

2. The method of claim 1, wherein localizing the pupil comprises measuring a displacement between at least two portions of the generated image.

3. The method of claim 2, wherein localizing the pupil further comprises, in response to determining that the at least two portions of the generated image are substantially aligned, determining that the pupil imaging device is positioned at a target position relative to the pupil.

4. The method of claim 3, further comprising capturing an image of a portion of a fundus of the subject's eye, using a fundus imaging device, upon determining that the pupil imaging device is positioned at the target position.

5. The method of claim 4, wherein an optical path of the fundus imaging device includes at least a portion of an optical path of the pupil imaging device.

6. The method of claim 5, further comprising providing feedback to a user and/or the subject indicating a current position of the pupil with respect to the target position.

7. The method of claim 6, wherein providing the feedback to the user and/or the subject comprises displaying, on a fixation target, the indication of the current position with respect to the target position.

8. The method of claim 1, further comprising capturing an image of a portion of a fundus of the subject's eye after localizing the pupil.

9. The method of claim 1, further comprising, after localizing the pupil, transmitting instructions to a fundus imaging device, wherein the instructions are configured to cause the fundus imaging device to capture an image of a portion of a fundus of the subject's eye.

10. The method of claim 1, further comprising, in response to localizing the pupil, transmitting instructions to the pupil imaging device, wherein the instructions are configured to cause a change in physical position of the pupil imaging device with respect to the pupil.

11. The method of claim 1, further comprising, prior to localizing the pupil, transmitting instructions to the pupil imaging device, wherein the instructions cause the pupil imaging device to illuminate the subject's eye.

12. The method of claim 1, wherein the split image component comprises a split prism configuration or a mirror configuration.

13. An imaging apparatus comprising:
a pupil imaging device comprising a split image component, wherein the pupil imaging device is configured to localize a pupil of a subject's eye in a field of view of the pupil imaging device using an image of at least a portion of the pupil, the image having been generated using light that was transmitted from the pupil through the split image component.

14. The imaging apparatus of claim 13, wherein the split image component comprises a prism configuration or a mirror configuration.

15. The imaging apparatus of claim 13, wherein the split image component is configured to receive light from an objective lens that is shared with the imaging apparatus.

16. The imaging apparatus of claim 13, wherein the imaging apparatus is configured for fundus imaging, optical imaging, optical coherence tomography (OCT), fluorescence imaging, color optical imaging, infrared (IR) imaging, and/or electro-optical imaging.

17. The imaging apparatus of claim 13, wherein the imaging apparatus is configured to provide feedback to a user in response to localizing the pupil, wherein the feedback is indicative of a current position of the pupil with respect to a target position.

18. The imaging apparatus of claim 13, further comprising one or more motors and/or actuators, wherein the one or more motors and/or actuators are configured to adjust a physical position of the imaging apparatus based on a result of the localizing.

19. The imaging apparatus of claim 18, wherein localizing the pupil comprises determining a distance between a current position of the pupil and a target position, and wherein the one or more motors and/or actuators are configured to adjust the physical position of the imaging apparatus to decrease the distance between the current position and the target position.
